(19) United States
(12) Patent Application Publication
(10) Pub. No.: US 2010/0233794 A1
(43) Pub. Date: Sep. 16, 2010

(54) SUCTION DEVICE AND ANALYSIS DEVICE

(76) Inventor: Kanako IWAMURA, Otawara-shi (JP)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/723,825

(22) Filed: Mar. 15, 2010

(30) Foreign Application Priority Data
Mar. 16, 2009 (JP) 2009-063250

(51) Int. Cl. C12M 1/34; B67D 99/00

(52) U.S. Cl. 435/287.3; 141/98

(57) ABSTRACT

Each of nozzles includes a micropore which is used as a channel, and the channel is exposed to the outside in the vicinity of a front end of the nozzle. An arm supports the nozzles so that the front ends of the nozzles are movable in an insertion/extraction direction, in which the front ends are inserted into or extracted from a specimen container, and the front ends of the nozzles are able to be simultaneously located in the specimen container. An arm movement mechanism and a nozzle movement mechanism respectively move the nozzles so as to move any one of the front ends of the nozzles in the insertion/extraction direction. Particularly, the nozzle movement mechanism is able to change a relative positional relationship of the front end of the nozzle movement mechanism with respect to the insertion/extraction direction.

### CROSS-REFERENCE TO RELATED APPLICATIONS

This application is based upon and claims the benefit of priority from prior Japanese Patent Application No. 2009-063250, filed Mar. 16, 2009, the entire contents of which are incorporated herein by reference.

### BACKGROUND

1. Field

The present invention relates to a suction device which sucks substances such as a blood plasma component and a blood cell component contained in a liquid substance such as blood stored in a container from the container, and an analysis device which analyzes characteristics of the liquid substance on the basis of the substances sucked by using the suction device.

2. Description of the Related Art

In a blood analysis using a blood analysis device, recently, a sample has been directly taken from a vacuum blood collection tube after a centrifuging operation in many cases. In addition, there is a case in which components in a blood corpuscle fraction are measured by using a vacuum blood collection tube containing anticoagulant (for example, refer to JP-A-2006-288220).

Incidentally, in the case where a blood plasma component and a blood cell component are measured in a totality of blood, it is necessary to suck the blood plasma component and the blood cell component from the totality of blood.

In this case, in the past, the blood plasma component and the blood cell component were separately sucked from the totality of blood, phase-separated into the blood plasma component and the blood cell component and stored in the container, by using one nozzle. However, in order to suck each of the blood plasma component and the blood cell component by using one nozzle, a cycle including a suction operation, a discharge operation, and a cleaning operation needs to be repeated twice for one subject. For this reason, much time is taken to reach the end of the operation of sucking the blood plasma component and the blood cell component. In addition, in the case where the nozzle is not sufficiently cleaned, one component remaining in the nozzle may be mixed with the other component.

### BRIEF SUMMARY OF THE INVENTION

An object of the invention is to suck a plurality of substances contained in a liquid substance in a short time while suppressing the mixture of the substances.

According to a first aspect of the invention, there is provided a suction device capable of sucking a substance contained in a liquid substance stored in a container, the suction device including: a plurality of nozzles each of which includes a channel and in which the channel is opened to the outside in the vicinity of a front end of the nozzle; a support member which supports the plurality of nozzles so that the front ends of the plurality of nozzles are moveable in an insertion/extraction direction, in which the front ends are inserted into or extracted from the container, and the front ends of the plurality of nozzles are able to be simultaneously located in the container; and a movement unit capable of changing a relative positional relationship of the front ends of the plurality of nozzles with respect to the insertion/extraction direction by moving the plurality of nozzles so as to move any one of the front ends of the plurality of nozzles in the insertion/extraction direction.

According to a second aspect of the invention, there is provided a suction device capable of sucking a substance contained in a liquid substance stored in a container, the suction device including: a plurality of nozzles each of which includes a channel and in which the channel is opened to the outside in the vicinity of a front end of the nozzle; a support member which fixes and supports the plurality of nozzles so that the front ends of the plurality of nozzles are able to be simultaneously located in the container and the entrance depths of the front ends of the plurality of nozzles with respect to the liquid substance are different from each other; and a movement unit which moves the support member so as to move the front ends of the plurality of nozzles in an insertion/extraction direction in which the front ends are inserted into or extracted from the container.

According to a third aspect of the invention, there is provided an analysis device including: a suction device according to the first or second aspect; and an analysis unit which analyzes different properties for the liquid substance on the basis of the substance sucked from the container by the plurality of nozzles included in the suction device.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing a configuration of an automatic analysis device according to an embodiment of the invention.

FIG. 2 is a perspective view showing a configuration of a sample section, a reagent section, and a reaction section of FIG. 1.

FIG. 3 is a partially cut away view showing a structure according to a first embodiment of a probe unit of FIG. 1.

FIG. 4 is a diagram showing a configuration of an electric circuit connected to a nozzle and a probe of FIG. 3.

FIG. 5 is a flowchart showing a process sequence according to the first embodiment of a system control unit of FIG. 1.

FIG. 6 is a diagram showing an example of the state where the further downward movement of an arm of FIG. 2 ends.

FIG. 7 is a diagram showing an example of the state where the downward movement of a nozzle using a nozzle movement mechanism stops.

FIG. 8 is a partially cut away view showing a structure according to a second embodiment of the probe unit of FIG. 1.

FIG. 9 is a flowchart showing a process sequence according to the second embodiment of the system control unit of FIG. 1.

### DETAILED DESCRIPTION

Hereinafter, several embodiments of the invention will be described with reference to the accompanying drawings.

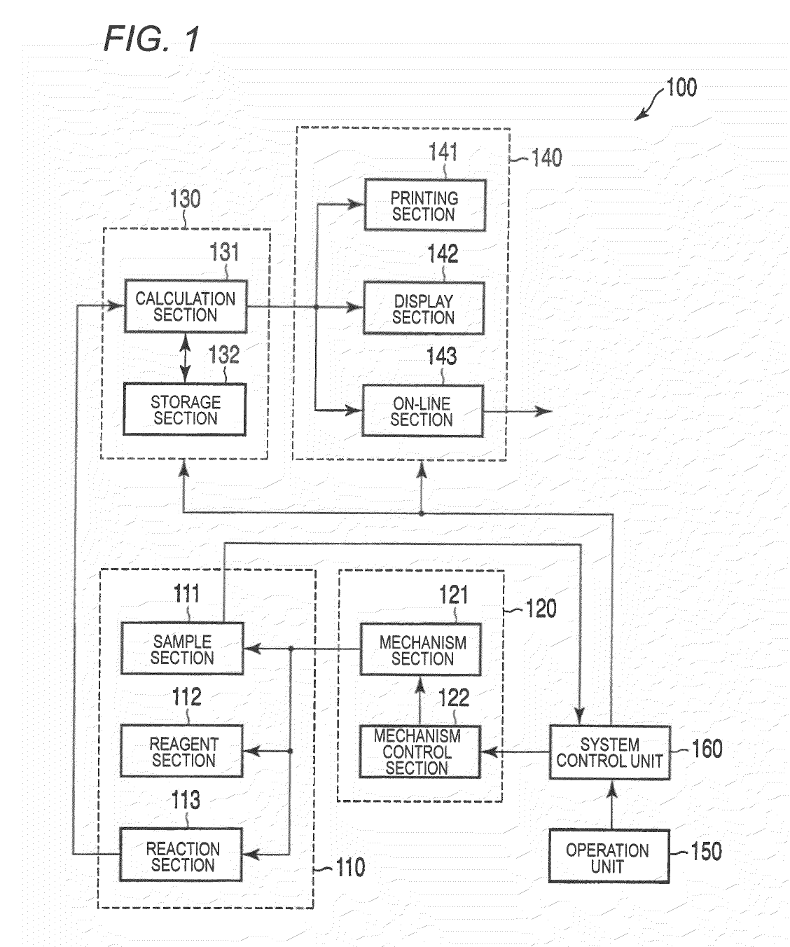

FIG. 1 is a block diagram showing a configuration of an automatic analysis device 100 according to the embodiments of the invention.

As shown in FIG. 1, the automatic analysis device 100 includes a measurement unit 110, an analysis control unit 120, an analysis data process unit 130, an output unit 140, an operation unit 150, and a system control unit 160.

The measurement unit 110 further includes a sample section 111, a reagent section 112, and a reaction section 113. The sample section 111 manages a calibrator for each measurement item or a test specimen (sample) taken from a subject. The reagent section 112 manages a reagent causing a chemical reaction with a component of the sample according to the measurement item. The reaction section 113 performs a measurement according to the measurement item for a reaction liquid between the sample and the reagent. The reaction section 113 outputs a calibrator signal indicating a measurement result for the calibrator and an analysis signal indicating the measurement result for the sample to the analysis data process unit 130.

The analysis control unit 120 further includes a mechanism section 121 and a mechanism control section 122. The mechanism section 121 drives various movable components described later and included in the measurement unit 110. The mechanism control section 122 controls the operation of the mechanism section 121.

The analysis data process unit 130 further includes a calculation section 131 and a storage section 132. The calculation section 131 creates a calibration table for each measurement item on the basis of the calibrator signal output from the measurement unit 110. The calculation section 131 calculates analysis data for each measurement item on the basis of the calibration table and the analysis signal output from the measurement unit 110. The storage section 132 includes a hard disk and the like, and stores the calibration table or the analysis data. The calculation section 131 outputs the calibration table or the analysis data to the output unit 140 if necessary.

The output unit 140 further includes a printing section 141, a display section 142, and an on-line section 143. The printing section 141 includes a printer or the like, and prints the analysis data or the calibration table output from the calculation section 131 on a printer sheet or the like in a predetermined format. The display section 142 includes a CRT (cathode-ray tube), an LCD (liquid crystal display), or the like, and displays the analysis data or the calibration table output from the calculation section 131 thereon.

Under the control of the system control unit 160, the display section 142 displays a subject information input screen for inputting a subject ID and a subject name and the like, an analysis condition setting screen for setting an analysis condition for each measurement item, a measurement item setting screen for selectively setting the measurement item for each sample, and the like. The on-line section 143 transmits the analysis data or the calibration table output from the calculation section 131 to another device via a network.

The operation unit 150 includes an input unit such as a keyboard, a mouse, a button, or a touch key panel. The operation unit 150 is operated by the operator so as to set the analysis condition for each measurement item, to input subject information such as a subject ID or a subject name, to selectively input the measurement item for each sample, and to perform the sample measurement or the calibration for each measurement item. The operation unit 150 outputs a command signal indicating the contents of the operation performed by the operator to the system control unit 160.

The system control unit 160 includes a CPU and a storage circuit, and generally controls the constituents of the automatic analysis device 100. In detail, the system control unit 160 determines the analysis condition for each measurement item, the subject information, the measurement item for each sample, and the like on the basis of the command signal supplied from the operation unit 150, and stores such information. The system control unit 160 controls the operation of the measurement unit 110 so as to perform the measurement in accordance with a predetermined sequence of a certain cycle on the basis of such information. The system control unit 160 controls the analysis data process unit 130 so as to calculate the desired analysis data or to create the desired calibration table. Further, the system control unit 160 controls the output unit 140 so as to output the calibration table or the analysis data in a desired format.

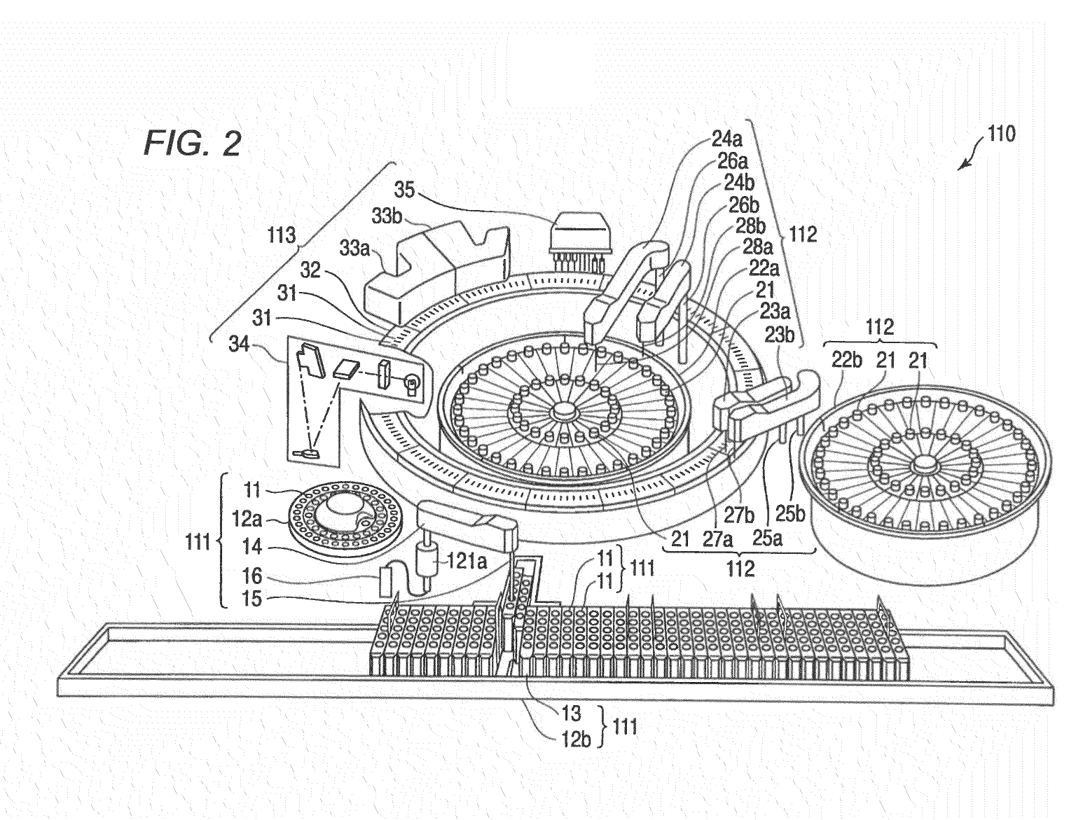

FIG. 2 is a perspective view showing a configuration of the sample section 111, the reagent section 112, and the reaction section 113.

The sample section 111 includes a reagent container 11, samplers 12a and 12b, a rack 13, an arm 14, a probe unit 15, and a pump unit 16.

The reagent container 11 stores a calibrator and a precise management specimen or sample.

The sampler 12a may be set in such a manner that a plurality of specimen containers 11 is disposed in two rows in a circumferential shape. The sampler 12a rotates to move the set specimen containers 11 along the circumference. Each position of setting the specimen containers 11 in the sampler 12a is allocated to a calibrator set position or a precise management specimen set position in advance. The specimen container 11 storing the calibrator is set in the former set position, and the specimen container 11 storing the precise management specimen is set in the latter set position.

The sampler 12b may be set in a plurality of the racks 13. The racks 13 may be set in such a manner that a plurality of the specimen containers 11 is arranged in a linear shape. The racks 13 are arranged along a direction perpendicular to the arrangement direction of the specimen containers 11. The sampler 12b moves the racks 13 in the arrangement direction thereof. In addition, the sampler 12b moves the racks 13 in a direction perpendicular to the arrangement direction thereof at the sample suction position. Each position where the specimen containers 11 are set in the racks 13 is allocated to the sample set position in advance, and the specimen container 11 storing the sample is set in the set position.

One end of an arm 14 is rotatably supported. The other end of the arm 14 is attached with a probe unit 15. The arm 14 is rotated by an arm movement mechanism 121a included in the mechanism section 121. In addition, the arm 14 is moved in the vertical direction by the arm movement mechanism 121a. In this manner, the arm 14 moves the probe unit 15 along the circular-arc orbit or moves the probe unit 15 in the vertical direction. The pump unit 16 generates a pressure for allowing the probe unit 15 to suck or discharge the sample in such a manner that the pump unit 16 sucks or discharges a pressure transfer medium such as water. In this manner, the arm 14, the probe unit 15, and the pump unit 16 constitute a suction device for sucking the sample stored in the specimen container 11.

The reagent section 112 includes a reagent bottle 21, reagent racks 22a and 22b, arms 23a, 23b, 24a, and 24b, leg portions 25a, 25b, 26a, and 26b, and reagent probes 27a, 27b, 28a, and 28b.

The reagent bottle 21 stores a reagent which selectively reacts with the sample.

The reagent racks 22a and 22b store a plurality of the reagent bottles 21, respectively. Each of the reagent racks 22a and 22b is a substantially circumferential container of which the upper surface is opened. The reagent racks 22a and 22b are capable of storing the plurality of reagent bottles 21 which are arranged in two rows in a circumferential shape, respectively. Each of the reagent racks 22a and 22b is rotated by a rotation mechanism not shown in FIG. 1 and described later.

One ends of the arms 23a, 23b, 24a, and 24b are respectively supported by the leg portions 25a, 25b, 26a, and 26b. The other ends of the arms 23a, 23b, 24a, and 24b are respectively attached with reagent probes 27a, 27b, 28a, and 28b.

When the leg portions 25a, 25b, 26a, and 26b are rotated by a known rotation mechanism not shown in FIG. 1, the arms 23a, 23b, 24a, and 24b are respectively rotated. In FIG. 1, a part of the leg portions 25a, 25b, 26a, and 26b is shown, and in practice they are longer than the shown size. In addition, the leg portions 25a, 25b, 26a, and 26b are linearly moved in the vertical direction by a known linear movement mechanism not shown in FIG. 1.

The reagent probes 27a, 27b, 28a, and 28b are moved along the circular-arc orbit or in the vertical direction by the arms 23a, 23b, 24a, and 24b and the leg portions 25a, 25b, 26a, and 26b. Each of the reagent probes 27a, 27b, 28a, and 28b has a cavity therein, and the cavity is connected to a pump (not shown) through the arms 23a, 23b, 24a, and 24b and the leg portions 25a, 25b, 26a, and 26b. The reagent probes 27a, 27b, 28a, and 28b suck or discharge the reagent by using a pressure generated by the pump connected thereto.

The reaction section 113 includes a reaction container 31, a disk 32, stirring units 33a and 33b, a side light unit 34, and a cleaning unit 35.

A plurality of the reaction containers 31 is arranged in a circumferential shape. The reaction containers 31 store the reaction liquid obtained by reacting the sample with the reagent.

A disk 32 rotatably holds the reaction containers 31. The disk 32 rotates in the counter-clockwise direction by a predetermined angle during four analysis cycles. One analysis cycle is, for example, 4.5 seconds. The disk 32 may rotate in the clockwise direction.

The stirring unit 33a includes two stirring members. The stirring unit 33a is capable of moving the two stirring members between two stirring positions corresponding to the upper position of the reaction container 31 and two cleaning positions different therefrom. In addition, the stirring unit 33a is capable of moving the two stirring members in the vertical direction. The stirring unit 33a has a function of cleaning each of the two stirring members at the two cleaning positions. The stirring unit 33a is used to stir a first reagent and the sample dispensed to the reaction container 31.

The stirring unit 33b includes two stirring members. The stirring unit 33b is capable of moving the two stirring members between two stirring positions corresponding to the upper position of the reaction container 31 and two cleaning positions different therefrom. In addition, the stirring unit 33b is capable of moving the two stirring members in the vertical direction. The stirring unit 33b has a function of cleaning each of the two stirring members at the two cleaning positions. The stirring unit 33b is used to stir a first reagent, a second reagent, and the sample dispensed to the reaction container 31.

The side light unit 34 emits light when the reaction container 31 passes the side light position, and measures a light absorption degree of a set wavelength on the basis of the transmitted light. In addition, the side light unit 34 generates an analysis signal as a signal indicating the measured light absorption degree.

The cleaning unit 35 includes a cleaning nozzle and a drying nozzle. The cleaning unit 35 sucks and cleans the reaction liquid in the inside of the reaction container 31 by using the cleaning nozzle. In addition, the cleaning unit 35 dries the inside of the reaction container 31 after the cleaning operation by using the drying nozzle. The reaction container 31 cleaned and dried by the cleaning unit 35 is used again for the measurement.

### First Embodiment

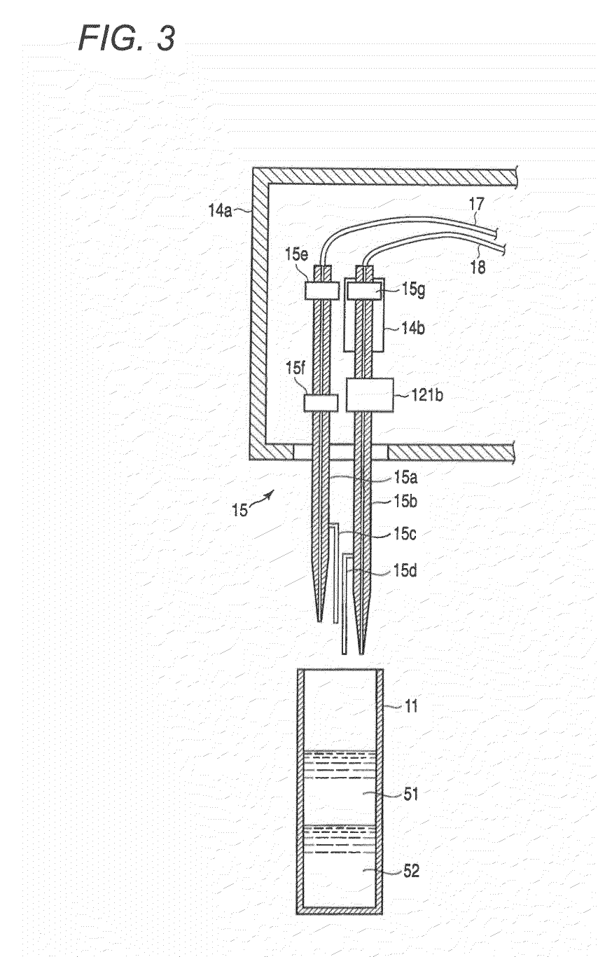

FIG. 3 is a partially cut away view showing a structure of the probe unit 15 according to a first embodiment.

The probe unit 15 includes nozzles 15a and 15b, probes 15c and 15d, and holding members 15e, 15f, and 15g.

The sections of the nozzles 15a and 15b are shown in FIG. 3. The external shape of each of the nozzles 15a and 15b is formed in a thin and long needle shape, and micropores are respectively formed therein so as to penetrate a portion between both ends. The micropores of the nozzles 15a and 15b are connected to the pump unit 16 through tubes 17 and 18. When the micropores of the nozzles 15a and 15b enter a negative pressure state by the pump unit 16, the nozzles 15a and 15b suck the sample into the micropores from the openings of the front ends. In addition, when the negative pressure inside the micropores is canceled by the pump unit 16, the nozzles 15a and 15b discharge the sample held inside the micropores. As a material of the nozzles 15a and 15b, for example, stainless steel or platinum is used, where the material has conductivity, is not deformed when the inside of the micropore enters a negative pressure, and is not degenerated due to the adhesion of the sample. In addition, the pump unit 16 has a function of individually controlling the pressure of each of the micropores of the nozzles 15a and 15b.

Each of the probes 15c and 15d is made from a conductive material not causing degeneration due to the adhesion to the sample, that is, stainless steel or platinum so as to have a thin and long bar shape. The probes 15c and 15d are respectively attached to the nozzles 15a and 15b so that the front end of the probe 15c has a predetermined gap with respect to the front end of the nozzle 15a and the front end of the probe 15d has a predetermined gap with respect to the front end of the nozzle 15b. In addition, in the portions of the nozzles 15a and 15b attached to the probes 15c and 15d, the nozzles 15a and 15b are insulated from the probes 15c and 15d.

The holding members 15e and 15f fix the nozzle 15a to a casing 14a of the arm 14 or a support member (not shown). The holding member 15g is fixed and attached to the nozzle 15b. The holding member 15g is attached to the casing 14a of the arm 14 or a guide portion 14b provided in a support member (not shown). The guide portion 14b supports the holding member 15g so as to be movable in the vertical direction (the lengthwise direction of FIG. 3).

In this manner, the relative position of the nozzle 15a with respect to the arm 14 cannot be changed, but the relative position of the nozzle 15b with respect to the arm 14 can be changed in the vertical direction. The nozzle 15b is held by a nozzle movement mechanism 121b. The nozzle movement mechanism 121b fixes the relative position of the nozzle 15b with respect to the arm 14 in the horizontal direction, and reciprocates the nozzle 15b in the vertical direction so that the relative position of the nozzle 15b with respect to the arm 14 in the vertical direction changes. The nozzle movement mechanism 121b may be directly configured as a known mechanism for reciprocating a bar-shaped object. In addition, the nozzle movement mechanism 121b is included in the mechanism section 121.

The horizontal relative position of the nozzles 15a and 15b and the probes 15c and 15d is set so as to be simultaneously inserted into one of the specimen containers 11. In addition, even in the state where the nozzle 15b is located at the uppermost side of the movable range, the vertical relative position of the nozzles 15a and 15b is set so that the front end of the nozzle 15b is located below the front end of the nozzle 15a.

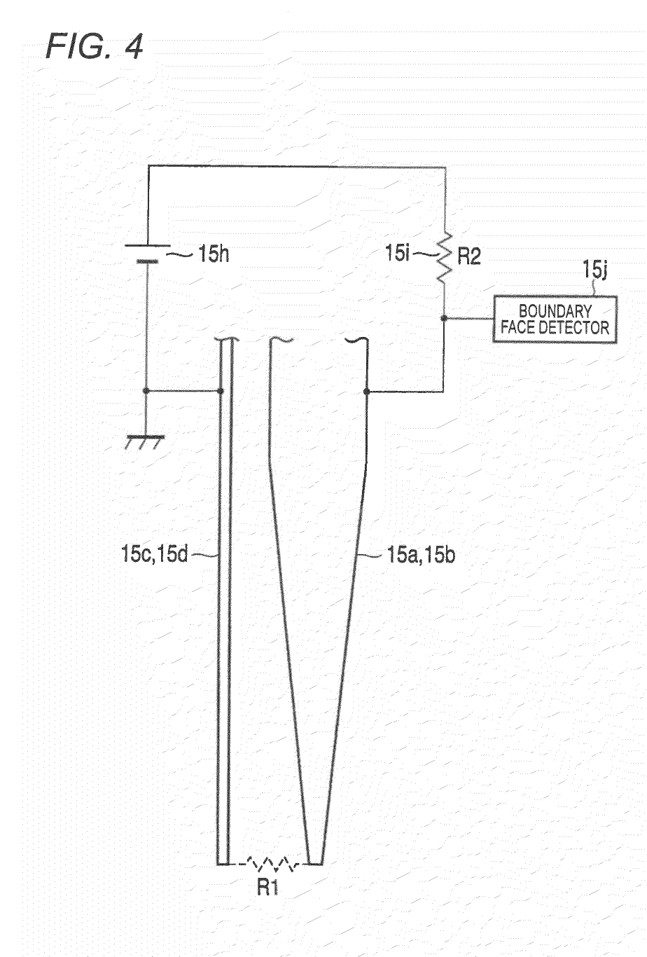

FIG. 4 is a diagram showing a configuration of an electric circuit connected to the nozzles 15a and 15b and the probes 15c and 15d. In addition, a pair of the electric circuits shown in FIG. 4 is provided in the nozzle 15a and the probe 15c, and a pair of the electric circuits is provided in the nozzle 15b and the probe 15d.

As shown in FIG. 4, the probe unit 15 includes an electric circuit including a power supply 15h, a resistor 15i, and a boundary face detector 15j in addition to the constituents shown in FIG. 3.

The power supply 15h and the resistor 15i are connected in series to each other between the nozzles 15a and 15b and the probes 15c and 15d. The boundary face detector 15j is connected to a connection point between the nozzles 15a and 15b and the resistor 15i. In this manner, in the electric circuit, a voltage value obtained by dividing an output voltage of the power supply 15h by an electric resistance value R1 of a substance existing between the nozzles 15a and 15b and the probes 15c and 15d and a resistance value of the resistor 15i is input to the boundary face detector 15j. The boundary face detector 15j detects a boundary face (hereinafter, referred to as a liquid face) between the external air and the sample or a boundary face (hereinafter, referred to as a boundary face) between a blood plasma component and a blood cell component inside the sample on the basis of a variation in the input voltage value. The detection result of the boundary face detector 15j is given to the system control unit 160.

Next, an operation of the automatic analysis device 100 including the probe unit 15 having the above-described configuration according to the first embodiment will be described. However, a characteristic operation of the automatic analysis device 100 according to the first embodiment is an operation of sucking blood from the specimen container 11 in the state where a blood plasma component and a blood cell component are in an interface separation state in the inside of the specimen container 11. Since the other operations are the same as those of the same type of existing automatic analysis device, description thereof is omitted.

As shown in FIG. 3, in the interface-separated blood, a blood plasma component 51 is located at the upper position, and a blood cell component 52 is located at the lower position. When it is necessary to dispense the blood plasma component 51 and the blood cell component 52 to the reaction container 31, the system control unit 160 positions the nozzles 15a and 15b at an upper position of the corresponding specimen container 11 by rotating the arm 14. In this state, the system control unit 160 performs a process of sucking the sample as below.

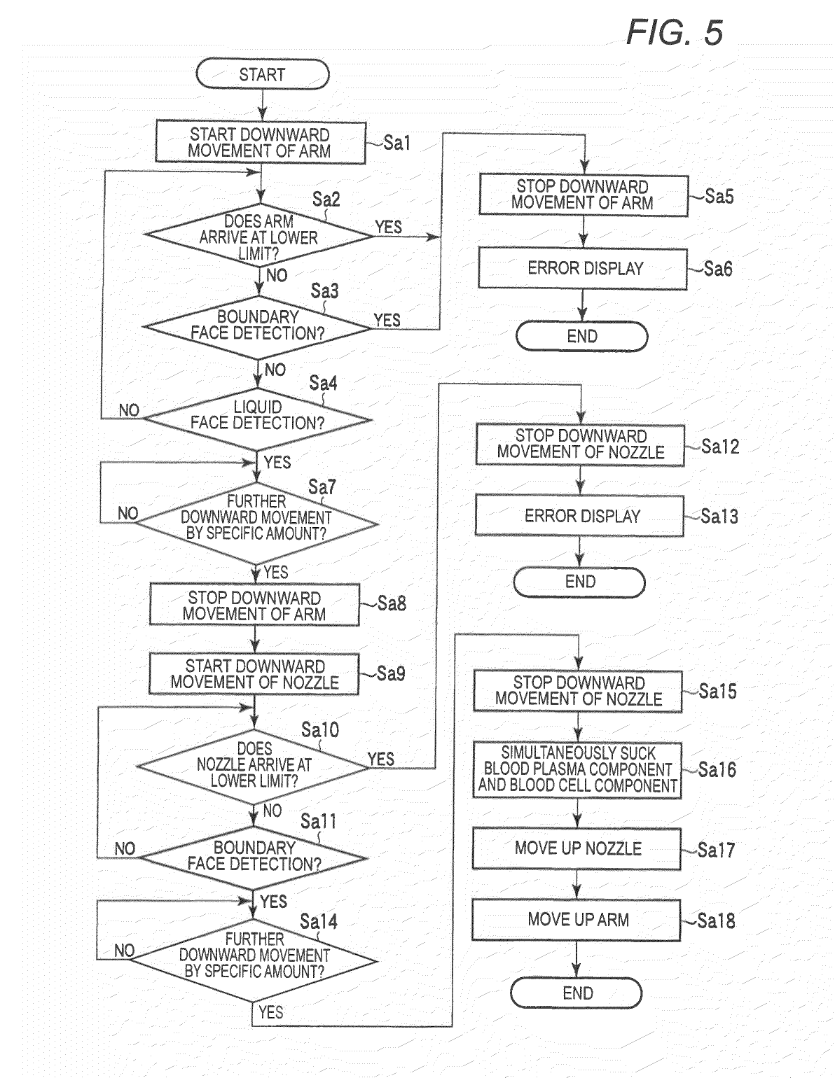

FIG. 5 is a flowchart showing a process sequence of the system control unit 160 when the sample is sucked from the specimen container 11.

In Step Sa1, the system control unit 160 instructs the mechanism control section 122 to move down the arm 14. In accordance with the downward movement of the arm 14, the nozzles 15a and 15b are moved down. In addition, at this time point, the nozzle 15b is located at the uppermost position (hereinafter, referred to as a reference position).

When the arm 14 is moved down in this manner, the nozzle 15b is first inserted into the specimen container 11, and then the nozzle 15a is inserted into the specimen container 11 after a while. When the arm 14 is further moved down, the nozzle 15b first arrives at the blood, and then the nozzle 15a arrives at the blood after a while.

Incidentally, when the front ends of the nozzles 15a and 15b do not arrive at the liquid face, since only the external air exists between the nozzles 15a and 15b and the probes 15c and 15d, the nozzles 15a and 15b and the probes 15c and 15d are insulated from each other. For this reason, a current does not flow to the resistor 15i, and a voltage is not input to the boundary face detector 15j.

When the front ends of the nozzles 15a and 15b arrive at the liquid face, the nozzles 15a and 15b and the probes 15c and 15d are electrically connected to each other through the blood plasma component 51. For this reason, a current flows to the resistor 15i, and a voltage is input to the boundary face detector 15j. In addition, when the front ends of the nozzles 15a and 15b arrive at the boundary face, since a substance electrically connecting the nozzles 15a and 15b and the probes 15c and 15d to each other changes from the blood plasma component 51 to a blood cell component 52, the resistance value R1 changes, and a voltage value input to the boundary face detector 15j changes. Therefore, the boundary face detector 15j connected to the nozzle 15a detects a state where the nozzle 15a arrives at the liquid face in accordance with a variation in voltage of the liquid face. In addition, the boundary face detector 15j connected to the nozzle 15b detects a state where the nozzle 15b arrives at the boundary face in accordance with a variation in voltage of the boundary face.

In the state where the arm 14 is moved down, the system control unit 160 enters the standby state from Step Sa2 to Step Sa4. In this standby state, the system control unit 160 waits for the state where the arm 14 arrives at a predetermined lower limit, the nozzle 15a arrives at the liquid face, or the nozzle 15b arrives at the boundary face.

Incidentally, in the case where a specific amount of sample is not stored in the specimen container 11, the arm 14 arrives at the lower limit before the nozzle 15a arrives at the liquid face. In addition, in the case where the amount of the blood plasma component 51 is small, the nozzle 15b arrives at the boundary face before the nozzle 15a arrives at the liquid face. In these cases, there is concern in that both the blood plasma component 51 and the blood cell component 52 are not correctly sucked. For this reason, in this case, the system control unit 160 moves the process from Step Sa2 or Step Sa3 to Step Sa5. In Step Sa5, the system control unit 160 instructs the mechanism control section 122 to stop the downward movement of the arm 14. Subsequently, in Step Sa6, the system control unit 160 instructs the display section 142 to perform an error display. Accordingly, the operator is informed that the sample is not correctly stored in the specimen container 11.

Meanwhile, when the system control unit 160 is in the standby state from Step Sa2 to Step Sa4, if it is detected that the nozzle 15a arrives at the liquid face, the system control unit 160 moves the process from Step Sa4 to Step Sa7. In Step Sa7, the system control unit 160 waits for the state where the arm 14 is further moved down by a specific amount after a time point when the nozzle 15a arrives at the liquid face. In addition, the further downward movement of the arm 14 is performed so as to insert the front end of the nozzle 15a into the blood plasma component 51 to a degree that the blood plasma component 51 is sufficiently sucked.

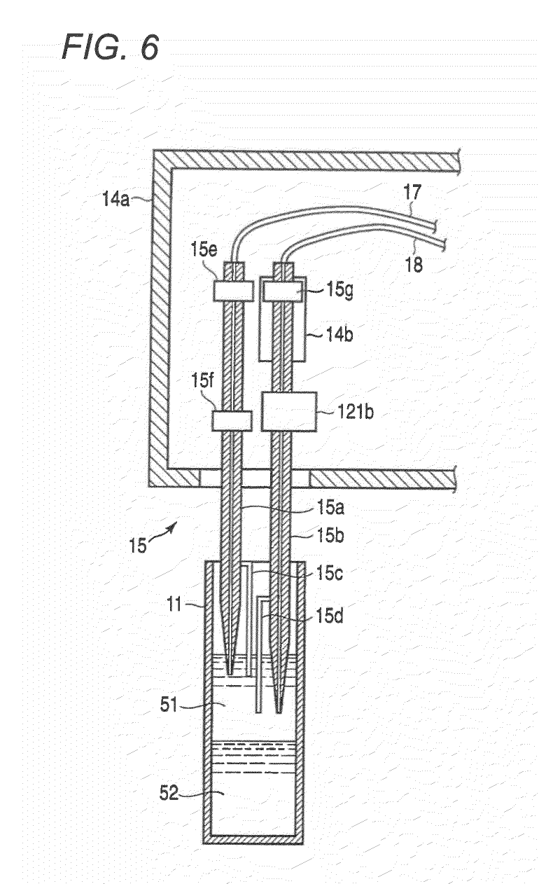

FIG. 6 is a diagram showing an example of the state where the further downward movement of the arm 14 ends.

When the further downward movement ends, the system control unit 160 moves the process from Step Sa1 to Step Sa8. In Step Sa8, the system control unit 160 instructs the mechanism control section 122 to stop the downward movement of the arm 14. Subsequently, in Step Sa9, the system control unit 160 instructs the mechanism control section 122 to start the operation of the nozzle movement mechanism 121b so that the nozzle 15b starts to move downward. In addition, in the state where the nozzle 15b moves down, the system control unit 160 enters the standby state of Step Sa10 and Step Sa11. In this standby state, the system control unit 160 waits for the state where the nozzle 15b arrives at the predetermined lower limit or the nozzle 15b arrives at the boundary face.

Incidentally, in the case where the amount of the blood cell component 52 is large, the nozzle 15b may arrive at the lower limit before the nozzle 15b arrives at the boundary face. Then, in this case, since it is not possible to insert the front end of the nozzle 15b into the blood cell component 52, it is not possible to correctly suck the blood cell component 52. Therefore, in this case, the system control unit 160 moves the process from Step Sa10 to Step Sa12. In Step Sa12, the system control unit 160 instructs the mechanism control section 122 to stop the downward movement of the nozzle 15b. Subsequently, in Step Sa13, the system control unit 160 instructs the display section 142 to perform an error display. Accordingly, the operator is informed that the sample is not correctly stored in the specimen container 11.

Meanwhile, when the system control unit 160 is in the standby state of Step Sa10 and Step Sa11, if it is detected that the nozzle 15b arrives at the boundary face, the system control unit 160 moves the process from Step Sa11 to Step Sa14. In addition, in Step Sa14, the system control unit 160 waits for the state where the nozzle 15b is further moved down by a specific amount after a time point when the nozzle 15b arrives at the liquid face. In addition, the further downward movement of the nozzle 15b is performed so as to insert the front end of the nozzle 15b into the blood cell component 52 to a degree that the blood cell component 52 is sufficiently sucked.

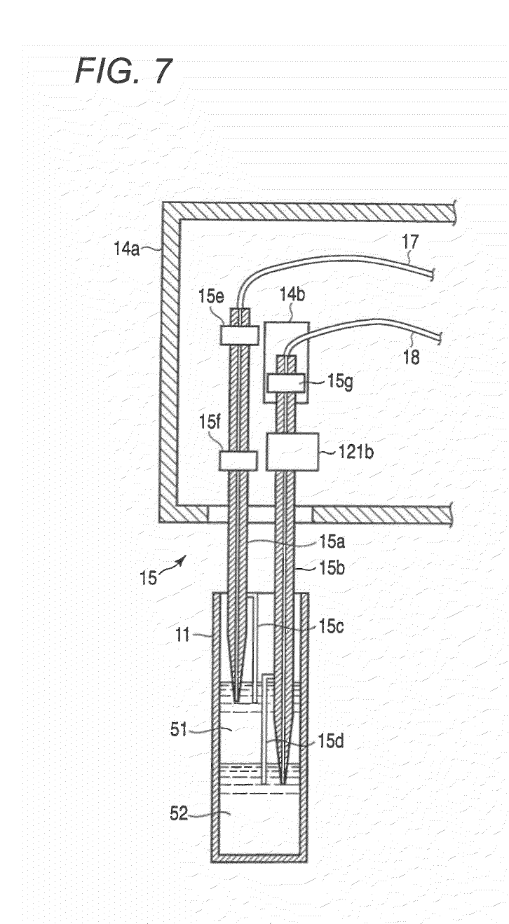

When the further downward movement ends, the system control unit 160 moves the process from Step Sa14 to Step Sa15. In Step Sa15, the system control unit 160 instructs the mechanism control section 122 to stop the downward movement of the nozzle 15b. Accordingly, as shown in FIG. 7, the nozzle 15a is stopped in the state where the front end thereof is inserted into the blood plasma component 51, and the nozzle 15b is stopped in the state where the front end thereof is inserted into the blood cell component 52. In Step Sa16, the system control unit 160 instructs the mechanism control section 122 to generate a negative pressure in the micropores of the nozzles 15a and 15b at the same time. Accordingly, the blood plasma component 51 and the blood cell component 52 are simultaneously sucked by the nozzles 15a and 15b.

Subsequently, in Step Sa17, the system control unit 160 instructs the mechanism control section 122 to operate the nozzle movement mechanism 121b so that the nozzle 15b is moved up to the reference position. Further, in Step Sa18, the system control unit 160 instructs the mechanism control section 122 to move up the arm 14 to the uppermost position.

As described above, according to the first embodiment, it is possible to simultaneously suck the blood plasma component 51 and the blood cell component 52. For this reason, it is possible to shorten the time required for the suction process by up to a half of the time required for the case where the operations of sucking the blood plasma component 51 and the blood cell component 52 are performed in time series. In addition, since the time required for the suction process is shortened, it is possible to shorten the time required for the inspection.

In addition, according to the first embodiment, since it is possible to change the relative positional relationship between the nozzles 15a and 15b in the vertical direction just by moving down the nozzle 15b, it is possible to respectively insert the front ends of the nozzles 15a and 15b into the blood plasma component 51 and the blood cell component 52 to a degree that the blood plasma component 51 and the blood cell component 52 are sufficiently sucked. As a result, the blood plasma component 51 and the blood cell component 52 can be precisely sucked.

### Second Embodiment

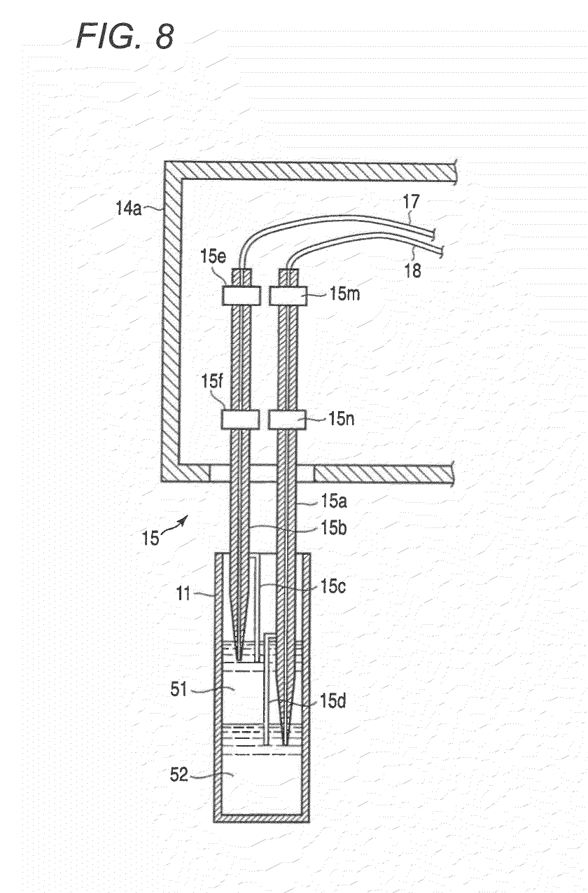

FIG. 8 is a partially cut away view showing a structure of the probe unit 15 according to a second embodiment. In addition, the same reference numerals are given to the same constituents as those of FIG. 3, and the detailed description thereof is omitted.

The probe unit 15 includes the nozzles 15a and 15b, the probes 15c and 15d, and the holding members 15e, 15f, 15m, and 15n.

That is, in the second embodiment, the probe unit 15 includes the holding members 15m and 15n instead of the holding member 15g according to the first embodiment. In addition, the guide portion 14b is not provided in the casing 14a, and the nozzle movement mechanism 121b is not included in the mechanism section 121.

The holding members 15m and 15n fix the nozzle 15b to the casing 14a or the support member (not shown).

The horizontal relative position of the nozzles 15a and 15b and the probes 15c and 15d is set so that they are simultaneously inserted into one of the specimen containers 11. In addition, the vertical relative position of the nozzles 15a and 15b is set so that the front end of the nozzle 15b is located below the front end of the nozzle 15a. Further, the vertical gap between the front end of the nozzle 15b and the front end of the nozzle 15a is set to be substantially equal to a standard gap between the liquid face and the boundary face.

Next, an operation of the automatic analysis device 100 including the probe unit 15 having the above-described configuration according to the second embodiment will be described. However, a characteristic operation of the automatic analysis device 100 according to the second embodiment is an operation of sucking blood from the specimen container 11 in the state where a blood plasma component and a blood cell component are in an interface separation state in the inside of the specimen container 11. Since the other operations are the same as those of the same type of existing automatic analysis device, description thereof is omitted.

The system control unit 160 rotates the arm 14 so that the nozzles 15a and 15b are located at the upper position of the specimen container 11 storing a blood as a suction target. In this state, the system control unit 160 performs the process for sucking the sample as below.

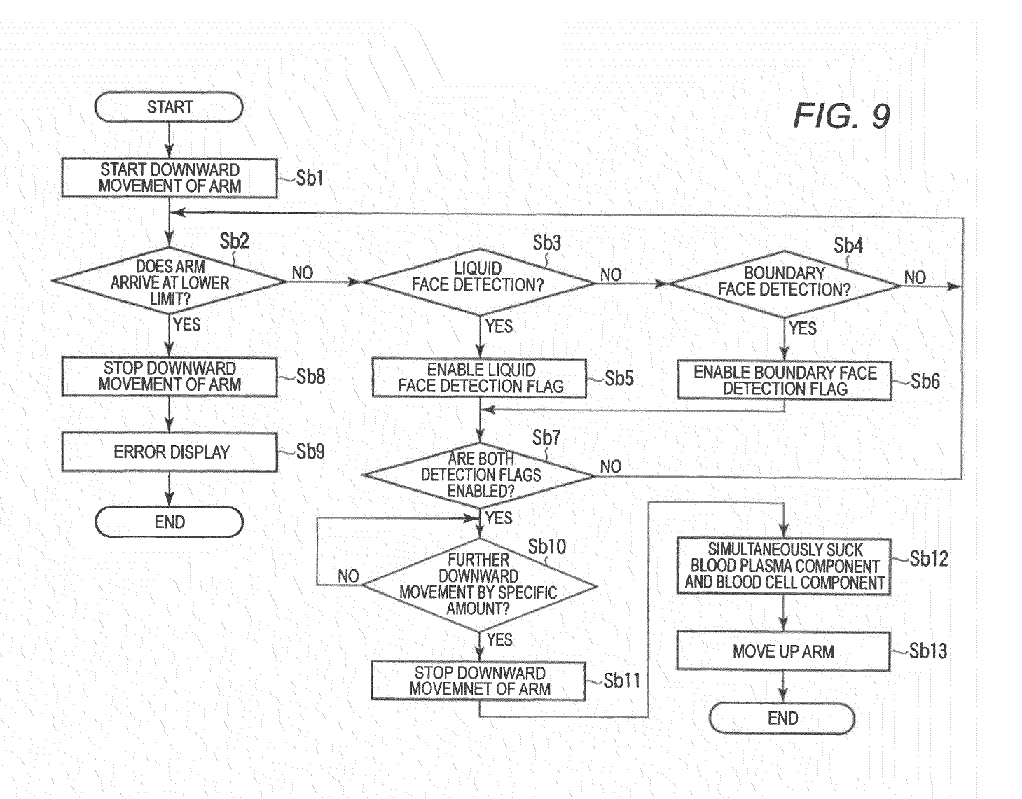

FIG. 9 is a flowchart showing a process sequence of the system control unit 160 when the sample is sucked from the specimen container 11.

In Step Sb1, the system control unit 160 instructs the mechanism control section 122 to move down the arm 14. In accordance with the downward movement of the arm 14, the nozzles 15a and 15b are moved down.

In the state where the arm 14 is moved down, the system control unit 160 enters the standby sate from Step Sb2 to Step Sb4. In this standby state, the system control unit 160 waits for the state where the arm 14 arrives at a predetermined lower limit, the nozzle 15a arrives at the liquid face, or the nozzle 15b arrives at the boundary face.

Incidentally, when the system control unit 160 is in the standby state from Step Sb2 to Step Sb4, if it is detected that the nozzle 15a arrives at the liquid face, the system control unit 160 moves the process from Step Sb3 to Step Sb5. Subsequently, in Step Sb5, the system control unit 160 enables a liquid face detection flag. Subsequently, the system control unit 160 moves the process to Step Sb7.

Meanwhile, when the system control unit 160 is in the standby state from Step Sb2 to Step Sb4, if it is detected that the nozzle 15b arrives at the boundary face, the system control unit 160 moves the process from Step Sb4 to Step Sb6. Then, in Step Sb6, the system control unit 160 enables a boundary face detection flag. Subsequently, the system control unit 160 moves the process to Step Sb7.

In addition, the liquid face detection flag and the boundary face detection flag are realized by, for example, a memory included in the system control unit 160. Further, the liquid face detection flag and the boundary face detection flag are disabled and initialized upon starting the process of FIG. 9.

In Step Sb7, the system control unit 160 checks whether both the liquid face detection flag and the boundary face detection flag are enabled. Then, when any one of the liquid face detection flag and the boundary face detection flag is disabled, the system control unit 160 returns to the standby state from Step Sb2 to Step Sb4.

Incidentally, in the case where any one of the blood plasma component 51 and the blood cell component 52 stored in the specimen container 11 is extremely small, the arm 14 arrives at the lower limit before the nozzle 15a arrives at the liquid face and the nozzle 15b arrives at the boundary face. Therefore, in this case, the system control unit 160 moves the process from Step Sb2 to Step Sb8. In Step Sb8, the system control unit 160 instructs the mechanism control section 122 to stop the downward movement of the arm 14. Subsequently, in Step Sb9, the system control unit 160 instructs the display section 142 to perform an error display. Accordingly, the operator is informed that the sample is not correctly stored in the specimen container 11.

Meanwhile, when the nozzle 15a arrives at the liquid face and the nozzle 15b arrives at the boundary face before the arm 14 arrives at the lower limit, the system control unit 160 is capable of checking that both the liquid face detection flag and the boundary face detection flag are enabled in Step Sb7. In this case, the system control unit 160 moves the process from Step Sb7 to Step Sb10. In Step Sb10, the system control unit 160 waits for the state where the arm 14 is further moved down by a specific amount from that time point. In addition, the further downward movement of the arm 14 is performed so as to insert the front ends of the nozzles 15a and 15b into the blood plasma component 51 and the blood cell component 52 to a degree that the blood plasma component 51 and the blood cell component 52 are sufficiently sucked. FIG. 8 is a diagram showing an example of the state where the further downward movement of the arm 14 ends.

When the further downward movement ends, the system control unit 160 moves the process from Step Sb10 to Step Sb11. In Step Sb11, the system control unit 160 instructs the mechanism control section 122 to stop the downward movement of the arm 14. Subsequently, in Step Sb12, the system control unit 160 instructs the mechanism control section 122 to simultaneously generate a negative pressure in the micropores of the nozzles 15a and 15b. Accordingly, the blood plasma component 51 and the blood cell component 52 are simultaneously sucked by the nozzles 15a and 15b.

Subsequently, in Step Sb13, the system control unit 160 instructs the mechanism control section 122 to move up the arm 14 to the uppermost position.

As described above, according to the second embodiment, it is possible to simultaneously suck the blood plasma component 51 and the blood cell component 52. For this reason, it is possible to shorten the time required for the suction process by up to a half of the time required for the case where the operations of sucking the blood plasma component 51 and the blood cell component 52 are performed in time series. In addition, since the time required for the suction process is shortened, it is possible to shorten the time required for the inspection.

Further, according to the second embodiment, since only the arm 14 is moved down so as to insert the nozzles 15a and 15b into a blood, it is possible to simplify the structure and control compared with the first embodiment. However, the adaptability for a difference in the amount of the blood plasma component 51 is higher in the first embodiment than the second embodiment.

The embodiments may be modified into various forms as below.

In the first embodiment, the nozzle 15a may be movable relative to the arm 14, and the nozzle 15b may be fixed to the arm 14. In addition, the nozzles 15a and 15b may be individually movable. Further, in the case where the nozzle 15a is movable, for example, the support structure and the nozzle movement mechanism which are the same as those of the nozzle 15b may be provided.

In the above-described embodiments, the movement direction of the nozzles 15a and 15b using the arm 14 and the movement direction of the nozzle 15b using the nozzle movement mechanism 121b are set to the vertical direction, but the movement direction of the nozzles 15a and 15b may be set to any direction in which the nozzles 15a and 15b are inserted into the specimen container 11 from the opening thereof.

In the above-described embodiments, the number of the nozzles included in the probe unit 15 may be three or more.

The substance as the suction target may be an arbitrary substance other than the blood plasma component 51 and the blood cell component 52, but may not be a blood component.

The probe unit 15 in the above-described embodiments may be applied to a device other than the automatic analysis device such as a blood inspection device.

A sensor for detecting a variation in the electric resistance value may be used instead of one probe so as to detect whether the nozzle arrives at the liquid face and the boundary face. Alternatively, the liquid face and the boundary face may be detected by using a variation in the capacitance, a variation in the pressure, or a variation in the light absorption degree.

When the component to be injected into the reaction container is any one of the blood plasma component 51 and the blood cell component 52, it is not necessary to move down all the nozzles 15a and 15b to be inserted into the specimen container 11. In such a case, for example, only the nozzle 15b is made to arrive at the necessary component, and the nozzle 15a is maintained to be exposed to the external air. For example, as shown in FIG. 7, when the nozzle 15b is inserted into the blood plasma component 51 while being moved down to the downmost position, the nozzle 15a can be maintained so as not to contact with any one of the blood plasma component 51 and the blood cell component 52.

That is, at least one of the nozzles is located in at least one of the plurality of regions divided by a plurality of the boundary faces so as to realize a state where the other nozzle is located in a space. In this manner, it is not necessary to perform a troublesome operation of cleaning the nozzle not contacting with the blood component.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A suction device capable of sucking a substance contained in a liquid substance stored in a container, the suction device comprising:
a plurality of nozzles each of which includes a channel and in which the channel is opened to the outside in the vicinity of a front end of the nozzle;
a support member which supports the plurality of nozzles so that the front ends of the plurality of nozzles are moveable in an insertion/extraction direction, in which the front ends are inserted into or extracted from the container, and the front ends of the plurality of nozzles are able to be simultaneously located in the container; and
a movement unit capable of changing a relative positional relationship of the front ends of the plurality of nozzles with respect to the insertion/extraction direction by moving the plurality of nozzles so as to move any one of the front ends of the plurality of nozzles in the insertion/extraction direction.

2. The suction device according to claim 1, further comprising:
a boundary face sensor which detects a boundary face between the liquid substance and external air and at least one boundary face generated by a phase separation of the liquid substance in the container,
wherein the movement unit moves the plurality of nozzles so that the front ends of the plurality of nozzles are located in a plurality of regions formed by the phase separation on the basis of a plurality of the boundary faces detected by the boundary face sensor.

3. The suction device according to claim 1, further comprising:
a boundary face sensor which detects a boundary face between the liquid substance and external air and at least one boundary face generated by a phase separation of the liquid substance in the container,
wherein the movement unit moves the plurality of nozzles so that the front end of at least one nozzle is located in at least one of a plurality of regions formed by the phase separation on the basis of a plurality of the boundary faces detected by the boundary face sensor.

4. A suction device capable of sucking a substance contained in a liquid substance stored in a container, the suction device comprising:
a plurality of nozzles each of which includes a channel and in which the channel is opened to the outside in the vicinity of a front end of the nozzle;
a support member which fixes and supports the plurality of nozzles so that the front ends of the plurality of nozzles are able to be simultaneously located in the container and the entrance depths of the front ends of the plurality of nozzles with respect to the liquid substance are different from each other; and
a movement unit which moves the support member so as to move the front ends of the plurality of nozzles in an insertion/extraction direction in which the front ends are inserted into or extracted from the container.

5. The suction device according to claim 4, further comprising:
a boundary face sensor which detects a boundary face between the liquid substance and external air and at least one boundary face generated by a phase separation of the liquid substance in the container,
wherein the movement unit moves the support member so that the front ends of the plurality of nozzles are located in a plurality of regions formed by the phase separation on the basis of a plurality of the boundary faces detected by the boundary face sensor.

6. The suction device according to claim 4, further comprising:
a boundary face sensor which detects a boundary face between the liquid substance and external air and at least one boundary face generated by a phase separation of the liquid substance in the container,
wherein the movement unit moves the support member so that the front end of at least one nozzle is located in at least one of a plurality of regions formed by the phase separation on the basis of a plurality of the boundary faces detected by the boundary face sensor.

7. An analysis device comprising:
a suction device according to claim 1; and
an analysis unit which analyzes different properties for the liquid substance on the basis of the substance sucked from the container by the plurality of nozzles included in the suction device.

8. An analysis device comprising:
a suction device according to claim 4; and
an analysis unit which analyzes different properties for the liquid substance on the basis of the substance sucked from the container by the plurality of nozzles included in the suction device.
